(19) United States
(12) Patent Application Publication
(10) Pub. No.: US 2006/0167013 A1
(43) Pub. Date: Jul. 27, 2006

(54) Inhibitors for excessive accumulation of sodium ion in cells

(76) Inventor: Naoya Satou, Tokyo (JP)

Correspondence Address: WENDEROTH, LIND & PONACK, L.L.P., 2033 K STREET N. W., SUITE 800, WASHINGTON, DC, 20006-1021, US

(21) Appl. No.: 10/525,980

(22) PCT Filed: Aug. 29, 2003
(86) PCT No.: PCT/JP03/11010

§ 371 Date: Sep. 20, 2005

(30) Foreign Application Priority Data
Aug. 30, 2002 (JP) 2002-255746

(51) Int. Cl. A61K 31/495

(52) U.S. Cl. 514/255.03

(57) ABSTRACT

A medicament for suppressing intracellular excess accumulation of sodium ions and a medicament for therapeutic and/or preventive treatment of cardiac disorders resulting from cardiosurgery operations which comprises an aminobenzenesulfonic acid derivative represented by the following general formula (I) or a salt thereof, or a hydrate thereof or a solvate thereof as an active ingredient.

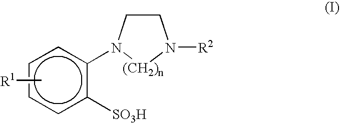

### TECHNICAL FIELD

The present invention relates to a suppressor of excess accumulation of intracellular sodium ions. The invention also relates to a medicament for therapeutic and/or preventive treatment of cardiac disorder accompanying cardiosurgery operations.

### BACKGROUND ART

In ischemic heart diseases such as myocardial infarction and angina, cessation of coronary artery blood flow for a certain period of time is followed by coronary artery reflow upon reperfusion therapy. Upon reperfusion, a huge amount of sodium ions flow into cardiac myocytes via pathways such as sodium channel, sodium/proton exchanger, and sodium/calcium exchanger, resulting in a condition of excess accumulation of sodium ions (hereinafter, sometimes referred to as “intracellular sodium overload”). It is known that the intracellular sodium overload successively induces excess accumulation of intracellular calcium ions (hereinafter sometimes referred to as “intracellular calcium overload” in the specification), mitochondrion dysfunction, cell membrane depolarization and the like, and thereby causing critical cardiac disorders such as myocardial contractile dysfunction, arrhythmia and the like (Cardio-vascular Research 2002; 55:141-149). In addition, when a cardiosurgery operation is conducted, it is common to establish a temporary cessation of blood flow to the heart, and therefore, it is known that a disorder resulting from ischemia and reperfusion occurs in the same manner as mentioned above.

Accordingly, it is expected that the suppression of the intracellular sodium overload upon ischemia and reperfusion may suppress myocardial disorder due to ischemia and reperfusion or cardiac disorders resulting from a cardiosurgery operation, and may possibly maintain cardiac function in a good condition. However, no medicament has been known so far which suppresses, in a clinically satisfactory manner, intracellular sodium overload due to ischemia and reperfusion.

Aminobenzenesulfonic acid derivatives are known which have a suppressing effect on excess accumulation of intracellular calcium ions in the cardiac muscle or the vascular smooth muscle (Japanese Patent Unexamined Publication (KOKAI) No. 3-7263). As for these compounds, the document discloses that they suppress or reduce cardiac disorders, heart conduction abnormalities and the like without beta receptor agonist-like effect, beta receptor antagonist-like effect, or calcium channel antagonist-like effect, and are expected to be useful agents for preventive or therapeutic treatment of ischemic heart diseases (for example, myocardial infarction, angina and the like), heart failure, hypertension, or arrhythmia (Japanese Patent Unexamined Publication (KOKAI) No. 3-7263 and Japanese Patent Unexamined Publication (KOKAI) No. 4-139127). Japanese Patent Unexamined Publication (KOKAI) No. 10-298077 discloses that the aforementioned compounds remarkably improve cardiac dysfunction under pathological condition of cardiomyopathy, and improve a long-term survival rate to achieve life lengthening in idiopathic cardiomyopathy. International publication WO 99/40919 discloses that the aforementioned compounds have promoting effect on uptake of calcium ions by sarcoplasmic reticulum in the cardiac muscle and are useful for therapeutic or preventive treatment of cardiac diastolic dysfunction.

However, these publications fails to teach whether these compounds suppress intracellular excess accumulation of sodium ions. As mentioned above, it is already known that these compounds suppress intracellular excess accumulation of calcium ions resulting from ischemia and reperfusion, but at present, it is not known whether these compounds suppress intracellular excess accumulation of sodium ions.

### DISCLOSURE OF THE INVENTION

An object of the present invention is to provide a medicament for suppressing intracellular excess accumulation of sodium ions, and a medicament for therapeutic and/or preventive treatment of cardiac disorders resulting from cardiosurgery operations.

The inventors of the present invention exerted various efforts to achieve the foregoing object, and as a result, they found that a specific class of aminobenzenesulfonic acid derivatives, salts thereof, hydrates thereof, or solvates thereof had suppressing effect on intracellular excess accumulation of sodium ions. The present invention was thus achieved.

The gist of the present invention resides in a medicament for suppressing intracellular excess accumulation of sodium ions which comprises an aminobenzenesulfonic acid derivative represented by the following formula (I): 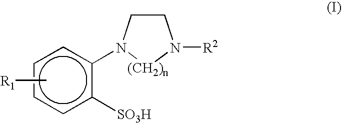
wherein R₁ represents hydrogen atom, a C₁-C₆ alkyl group, a C₃-C₇ cycloalkyl group, a halogenated C₁-C₄ alkyl group, a halogen atom or a C₆-C₁₂ aryl group; R₂ represents hydrogen atom, a C₁-C₆ alkyl group or a C₇-C₁₂ aralkyl group which may have one or more substituents selected from the group consisting of cyano group, nitro group, a C₁-C₆ alkoxyl group, a halogen atom, a C₁-C₆ alkyl group, and an amino group; and n represents an integer of 1 to 4, or a salt thereof, or a hydrate thereof or a solvate thereof as an active ingredient.

According to preferred embodiments, the aforementioned medicament for suppressing intracellular excess accumulation of sodium ions is that for therapeutic and/or preventive treatment of disorders resulting from ischemia and reperfusion; and the aforementioned medicament for suppressing intracellular excess accumulation of sodium ions is characterized as a suppressor against an increase of a sodium ion content in cardiac myocytes induced by disorders resulting from ischemia and reperfusion.

From another aspect, the present invention relates to a medicament for therapeutic and or preventive treatment of diseases caused by intracellular excess accumulation of sodium ions (provided that an ischemic heart disease, cardiac failure, hypertension, and arrhythmia are excluded); and cardiovascular diseases resulting from intracellular excess accumulation of calcium ions which is induced successively after intracellular excess accumulation of sodium ions, which comprises the aforementioned medicament for suppressing intracellular excess accumulation of sodium ions as an active ingredient.

From further aspect, the present invention relates to a medicament for therapeutic and/or preventive treatment of cardiac disorders resulting from cardiosurgery operations which comprises the aminobenzenesulfonic acid derivative represented by the aforementioned general formula (I) or the like as an active ingredient.

According to the present invention, a medicament for suppressing intracellular excess accumulation of sodium ions, and a medicament for therapeutic and/or preventive treatment of cardiac disorders resulting from cardiosurgery operations are provided. As demonstrated by a working example set out below, the compounds represented by the aforementioned general formula (I) have suppressing action against intracellular excess accumulation of sodium ions, and accordingly, they are useful for therapeutic and/or preventive treatment of disorders resulting from ischemia and reperfusion. Further, the compounds represented by the aforementioned general formula (I) have suppressing action against intracellular excess accumulation of sodium ions, and accordingly, they are effective for therapeutic and/or preventive treatment of diseases resulting from intracellular excess accumulation of sodium ions (provided that ischemic heart disease, heart failure, hypertension, and arrhythmia are excluded). In addition, because the intracellular excess accumulation of sodium ions induces intracellular excess accumulation of calcium ions, they are effective for therapeutic and/or preventive treatment of diseases resulting from intracellular excess accumulation of calcium ions, which is induced successively after intracellular excess accumulation of sodium ions, more specifically, an ischemic heart disease, heart failure, hypertension, and arrhythmia, for example. Specific examples of the ischemic heart disease include, for example, myocardial infarction and angina.

Examples of the active ingredient of the medicament of the present invention includes the aminobenzenesulfonic acid derivatives represented by the aforementioned formula (I) or salts thereof, or hydrates thereof or solvates thereof. In the aforementioned formula (I), examples of the C₁-C₆ alkyl group defined by R₁ include, for example, methyl group, ethyl group, propyl group, isopropyl group, butyl group, isobutyl group, sec-butyl group, tert-butyl group, pentyl group, isopentyl group, neopentyl group, tert-pentyl group, hexyl group, isohexyl group and the like. Examples of the C₃-C₇ cycloalkyl group include cyclopropyl group, cyclobutyl group, cyclopentyl group, cyclohexyl group, cycloheptyl group and the like. Examples of the halogenated C₁-C₄ alkyl group include, for example, trifluoromethyl group, trifluoroethyl group, pentafluoroethyl group and the like. Examples of the halogen atom include, for example, fluorine atom, chlorine atom, bromine atom and the like. Examples of the C₆-C₁₂ aryl group include, for example, phenyl group, naphthyl group and the like.

Preferred examples of R₁ include hydrogen atom, a C₁-C₆ alkyl group, a C₅-C₆ cycloalkyl group, trifluoromethyl group, a halogen atom, and phenyl group, and more preferred examples of R₁ are a C₁-C₃ alkyl group, cyclohexyl group, trifluoromethyl group, chlorine atom, bromine atom, and phenyl group. Particularly preferred are methyl group and propyl group. A position of substitution with R₁ is preferably 5-position.

Examples of the C₁-C₆ alkyl group defined by R₂ include, for example, the alkyl groups defined above as R₁. Examples of the C₇-C₁₂ aralkyl group include, for example, benzyl group, phenethyl group, naphthylmethyl group and the like. This aralkyl group may have one or more substituents selected from the group consisting of cyano group; nitro group; a C₁-C₆ alkoxy group such as methoxy group, ethoxy group, propoxy group, isopropoxy group, butoxy group, isobutoxy group, tert-butoxy group, pentyloxy group, isopentyloxy group, tert-pentyloxy group, and hexyloxy group; a halogen atom such as those defined in the above R₁; an alkyl group such as those defined in the above R₁, and an amino group.

Preferred examples of R₂ include hydrogen atom, a C₁-C₃ alkyl group and a C₇-C₁₂ aralkyl group which may have one or more substituents selected from a C₁-C₃ alkyl group, a C₁-C₃ alkoxyl group, and a halogen atom, and more preferred examples of R₂ include hydrogen atom and a C₇-C₁₂ aralkyl group which may have one or more substituents selected from C₁-C₃ alkoxyl groups. Particularly preferred is hydrogen atom.

In the aforementioned formula (I), n is preferably 2.

Specific examples according to the present invention include the compounds shown in Tables 1 and 2 set out below.

TABLE 1

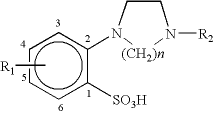

| | Substituting | | | |
|---|---|---|---|---|
| Compound | position | | | |
| No. | of R₁ | R₁ | n | R₂ |
| 1 | — | H | 2 | H |
| 2 | 3 | —CH₃ | 2 | H |
| 3 | 3 | —CH₂CH₃ | 2 | H |
| 4 | 3 | —CH₂CH₂CH₃ | 2 | H |
| 5 | 3 | —CH(CH₃)₂ | 2 | H |
| 6 | 3 | —(CH₂)₃CH₃ | 2 | H |
| 7 | 4 | —CH₃ | 2 | H |
| 8 | 4 | —CH₂CH₃ | 2 | H |
| 9 | 4 | —(CH₂)₂CH₃ | 2 | H |
| 10 | 4 | —CH(CH₃)₂ | 2 | H |
| 11 | 4 | —(CH₂)₃CH₃ | 2 | H |
| 12 | 5 | —CH₃ | 2 | H |
| 13 | 5 | —CH₂CH₃ | 2 | H |
| 14 | 5 | —(CH₂)₂CH₃ | 2 | H |
| 15 | 5 | —CH(CH₃)₂ | 2 | H |
| 16 | 5 | —(CH₂)₃CH₃ | 2 | H |
| 17 | 5 | —(CH₂)₄CH₃ | 2 | H |
| 18 | 5 | —(CH₂)₅CH₃ | 2 | H |
| 19 | 6 | —CH₃ | 2 | H |
| 20 | 6 | —CH₂CH₃ | 2 | H |
| 21 | 6 | —(CH₂)₂CH₃ | 2 | H |
| 22 | — | H | 2 | —CH₃ |
| 23 | 3 | —CH₂CH₃ | 2 | —CH₃ |
| 24 | 3 | —(CH₂)₂CH₃ | 2 | —CH₃ |
| 25 | 3 | —CH(CH₃)₂ | 2 | —CH₃ |
| 26 | 3 | —(CH₂)₃CH₃ | 2 | —CH₃ |
| 27 | 4 | —CH₃ | 2 | —CH₃ |
| 28 | 4 | —CH₂CH₃ | 2 | —CH₃ |
| 29 | 4 | —(CH₂)₂CH₃ | 2 | —CH₃ |
| 30 | 5 | —CH₃ | 2 | —CH₃ |
| 31 | 5 | —CH₂CH₃ | 2 | —CH₃ |
| 32 | 5 | —(CH₂)₂CH₃ | 2 | —CH₃ |
| 33 | 5 | —CH(CH₃)₂ | 2 | —CH₃ |
| 34 | 5 | —(CH₂)₃CH₃ | 2 | —CH₃ |
| 35 | 5 | —(CH₂)₄CH₃ | 2 | —CH₃ |
| 36 | 5 | —(CH₂)₅CH₃ | 2 | —CH₃ |
| 37 | 6 | —CH₃ | 2 | —CH₃ |
| 38 | 6 | —CH₂CH₃ | 2 | —CH₃ |
| 39 | 6 | —(CH₂)₂CH₃ | 2 | —CH₃ |
| 40 | 6 | —OH(CH₃)₂ | 2 | —CH₃ |
| 41 | 6 | —(CH₂)₃CH₃ | 2 | —CH₃ |
| 42 | 3 | —(CH₂)₂CH₃ | 2 | —(CH₂)₂CH₃ |
| 43 | 4 | —(CH₂)₂CH₃ | 2 | —(CH₂)₂CH₃ |
| 44 | 5 | —CH₃ | 2 | —(CH₂)₂CH₃ |
| 45 | 5 | —CH₂CH₃ | 2 | —(CH₂)₂CH₃ |
| 46 | 5 | —(CH₂)₂CH₃ | 2 | —(CH₂)₂CH₃ |
| 47 | 5 | —CH(CH₃)₂ | 2 | —(CH₂)₂CH₃ |
| 48 | 5 | —(CH₂)₃CH₃ | 2 | —(CH₂)₂CH₃ |
| 49 | 5 | —(CH₂)₅CH₃ | 2 | —(CH₂)₂CH₃ |
| 50 | — | H | 2 | —(CH₂)₂CH₃ |
| 51 | — | H | 2 | 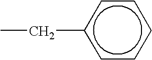 |
| 52 | 3 | —CH₃ | 2 | 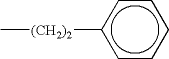 |
| 53 | 3 | —(CH₂)₂CH₃ | 2 | 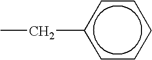 |
| 54 | 4 | —CH₃ | 2 | 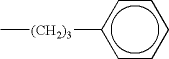 |
| 55 | 4 | —(CH₂)₂CH₃ | 2 | 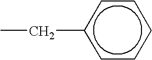 |
| 56 | 5 | —CH₃ | 2 | 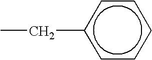 |
| 57 | 5 | —CH₂CH₃ | 2 | 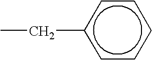 |
| 58 | 5 | —(CH₂)₂CH₃ | 2 | 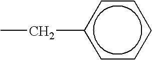 |
| 59 | 5 | —CH(CH₃)₂ | 2 | 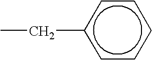 |
| 60 | 5 | —(CH₂)₃CH₃ | 2 | 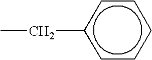 |
| 61 | 5 | —(CH₂)₄CH₃ | 2 | 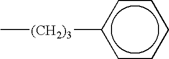 |
| 62 | 5 | —(CH₂)₂CH₃ | 2 | 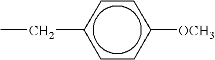 |
| 63 | 5 | —CH(CH₃)₂ | 2 | 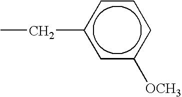 |
| 64 | 5 | —CH(CH₃)₂ | 2 | 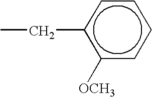 |
| 65 | 4 | —(CH₂)₂CH₃ | 2 | 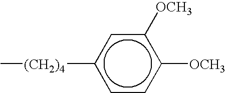 |
| 66 | 5 | —(CH₂)₂CH₃ | 2 | 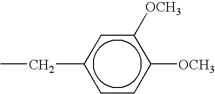 |
| 67 | 5 | —CH(CH₃)₂ | 2 | 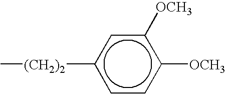 |
| 68 | 6 | —(CH₂)₂CH₃ | 2 | 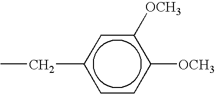 |
| 69 | 5 | —(CH₂)₂CH₃ | 2 | 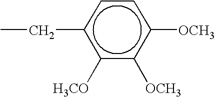 |
| 70 | 6 | —(CH₂)₂CH₃ | 2 | 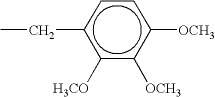 |
| 71 | 3 | —(CH₂)₂CH₃ | 2 | 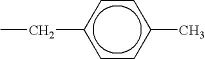 |
| 72 | 4 | —(CH₂)₂CH₃ | 2 | 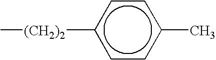 |
| 73 | 5 | —(CH₂)₂CH₃ | 2 | 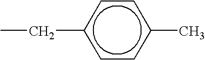 |
| 74 | 6 | —CH(CH₃)₂ | 2 | 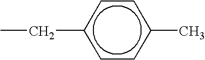 |
| 75 | 3 | —(CH₂)₂CH₃ | 2 | 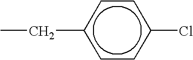 |
| 76 | 4 | —(CH₂)₂CH₃ | 2 | 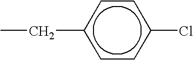 |
| 77 | 5 | —(CH₂)₂CH₃ | 2 | 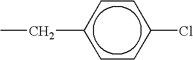 |
| 78 | 6 | —(CH₂)₂CH₃ | 2 | 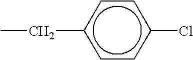 |
| 79 | 3 | —(CH₂)₂CH₃ | 2 | 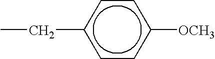 |
| 80 | 4 | —(CH₂)₂CH₃ | 2 | 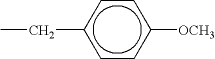 |
| 81 | 5 | —(CH₂)₂CH₃ | 2 | 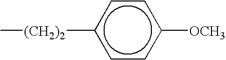 |
| 82 | 6 | —(CH₂)₂CH₃ | 2 | 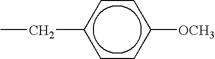 |
| 83 | — | H | 3 | H |
| 84 | 5 | —CH₃ | 3 | H |
| 85 | 5 | —CH₂CH₃ | 3 | H |
| 86 | 5 | —(CH₂)₂CH₃ | 3 | H |
| 87 | 5 | —CH(CH₃)₂ | 3 | H |
| 88 | 5 | —(CH₂)₂CH₃ | 3 | H |
| 89 | 5 | —(CH₂)₂CH₃ | 3 | —CH₃ |
| 90 | 5 | —(CH₂)₂CH₃ | 3 | 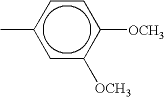 |
| 91 | 5 | 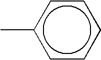 | 2 | H |
| 92 | 5 | —F | 2 | H |
| 93 | 5 | —Cl | 2 | H |
| 94 | 5 | —Br | 2 | H |
| 95 | 5 | —CF₃ | 2 | H |
| 96 | 5 | 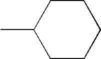 | 2 | H |
| 97 | 5 | 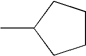 | 2 | H |
| 98 | 5 | 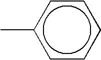 | 2 | —CH₃ |
| 99 | 5 | —Cl | 2 | —CH₃ |
| 100 | 5 | —Br | 2 | —CH₃ |
| 101 | 5 | —CF₃ | 2 | —CH₃ |
| 102 | 5 | 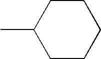 | 2 | —CH₃ |
| 103 | 5 | 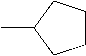 | 2 | —CH₃ |
| 104 | 5 | 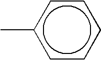 | 2 | 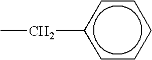 |
| 105 | 5 | —Cl | 2 | 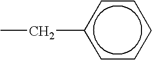 |
| 106 | 5 | —Br | 2 | 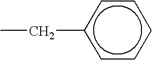 |
| 107 | 5 | —CF₃ | 2 | 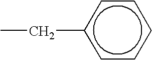 |
| 108 | 5 | 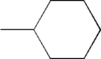 | 2 | 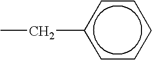 |
| 109 | 5 | 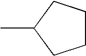 | 2 | 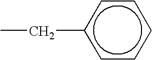 |

TABLE 2

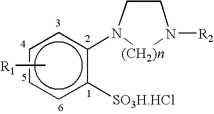

| | Substituting | | | |
|---|---|---|---|---|
| Compound | position | | | |
| No. | of R₁ | R₁ | n | R₂ |
| 110 | 5 | —CH₂CH₂CH₃ | 2 | H |
| 111 | 5 | —CH(CH₃)₂ | 2 | H |
| 112 | 5 | 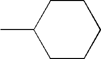 | 2 | H |
| 113 | 5 | 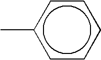 | 2 | H |
| 114 | 5 | —Cl | 2 | H |
| 115 | 5 | —Br | 2 | H |
| 116 | 5 | —CF₃ | 2 | H |

Among the compounds shown in Tables 1 and 2 set out above, the compounds wherein the position of substitution with R₁ is 5-position are preferred, and further preferred compounds include the following compounds:
5-methyl-2-(1-piperazinyl)benzenesulfonic acid;
5-trifluoromethyl-2-(1-piperazinyl)benzenesulfonic acid;
5-n-propyl-2-(1-piperazinyl)benzenesulfonic acid;
5-phenyl-2-(1-piperazinyl)benzenesulfonic acid;
5-chloro-2-(1-piperazinyl)benzenesulfonic acid;
5-bromo-2-(1-piperazinyl)benzenesulfonic acid;
5-iso-propyl-2-(1-piperazinyl)benzenesulfonic acid;
5-cyclohexyl-2-(1-piperazinyl)benzenesulfonic acid;
5-n-propyl-2-(1-homopiperazinyl)benzenesulfonic acid;
5-n-propyl-2-[4-(2,3,4-trimethoxybenzyl)- 1-piperazinyl]benzenesulfonic acid;
5-n-propyl-2-[4-(3,4-dimethoxybenzyl)-1-piperazinyl]benzenesulfonic acid.

Among the aforementioned compounds, particularly preferred examples include 5-methyl-2-(1-piperazinyl)benzenesulfonic acid and 5-n-propyl-2-(1-piperazinyl)benzenesulfonic acid.

Pharmacologically acceptable salts of the compounds mentioned above also fall within the scope of the present invention. Examples of the salts of the aforementioned compounds include, for example, alkali metal salts and alkaline earth metal salts such as sodium salts, potassium salts, magnesium salts, calcium salts, and aluminum salts; amine salts, for example, ammonium salts, lower alkylamine salts such as triethylamine salts, hydroxy(lower alkyl)amine salts such as 2-hydroxyethylamine salts, bis(2-hydroxyethyl)amine salts, tris(hydroxymethyl)aminomethane salts, and N-methyl-D-glucamine salts, cycloalkylamine salts such as dicyclohexylamine salts, benzylamine salts and dibenzylamine salts such as N,N-dibenzylethylenediamine salts; inorganic acid salts such as hydrochlorides, hydrobromides, sulfates, and phosphates; organic acid salts such as fumarates, succinates, oxalates, lactates and the like.

Besides the salts or the compounds in free forms, any hydrates or solvates thereof may also be used as active ingredients of the medicaments of the present invention. Examples of solvents that can form solvates of the aforementioned compounds include, for example, methanol, ethanol, isopropyl alcohol, acetone, ethyl acetate, methylene chloride and the like.

A most preferred example of the active ingredient of the medicament of the present invention includes 5-methyl-2-(1-piperazinyl)benzenesulfonic acid monohydrate.

The aminobenzenesulfonic acid derivatives represented by the aforementioned general formula (I) are known compounds, and they are easily synthesizable and readily available to those skilled in the art by the methods described in, for example, Japanese Patent Unexamined Publication (KOKAI) Nos. (Hei)3-7263 and (Hei)9-221479, European Patent Publication Nos. 390654 and 779283, U.S. Pat. Nos. 5,053,409 and 5,990,113 and the like.

The therapeutic and/or preventive medicament of the present invention can be orally or parenterally administered to a human in an ordinary manner. Examples of formulations for oral administration include granules, subtilized granules, powders, tablets, hard capsules, soft capsules, syrups, emulsions, suspensions, solutions and the like. Examples of formulations for parenteral administration include injections, suppositories, transdermal preparations and the like.

The active ingredient of the present invention is contained in the aforementioned formulations together with a solid or liquid pharmaceutical carrier, or an ordinarily used pharmaceutical additive such as an excipient, a stabilizer, a lubricant, a sweetening agent, a preservative, a suspending aid and the like. A content ratio of the therapeutic or preventive active ingredient relative to the ingredient as the pharmaceutical carrier may preferably be 1 to 90% by weight.

Examples of usable solid ingredients include lactose, kaolin, sucrose, crystalline cellulose, cornstarch, talc, agar, pectin, acacia, stearic acid, magnesium stearate, lecithin, sodium chloride and the like. Examples of liquid carriers include syrup, glycerol, peanut oil, polyvinylpyrrolidone, olive oil, ethanol, benzyl alcohol, propylene glycol, water and the like.

A dose of the substance used as the active ingredient may be appropriately determined, depending on a kind of the active ingredient, in view of a purpose of treatment or prevention, a kind of a disease to be treated or prevented, symptoms, body weight, age, and sexuality of a patient and the like. As for the compound represented by the aforementioned general formula (I) which is considered as a typical example, a dose of about 0.01 to 1,000 mg per day can generally be administered orally to an adult. The above dose may preferably be administered once a day or several times a day as divided portions.

### EXAMPLE

The present invention will be more specifically explained by referring to an example. However, the present invention is not limited to the following examples as long as it does not go beyond the scope thereof.

In the compound of the present invention referred to in the following example is 5-methyl-2-(1-piperazinyl)benzenesulfonic acid monohydrate (hereinafter occasionally referred to as “MCC-135”). The substance used was prepared according to Example 1 of Japanese Patent Unexamined Publication (KOKAI) No. 9-221479.

### Example 1

(Experimental Methods)

The heart of the rat was excised and perfused with Krebs buffer solution (in mM: NaCl 119, KCl 4.6, MgSO₄.7H₂O 1.2, CaCl₂·2H₂O 1.3, NaHCO₃ 25, KH₂PO₄ 1.2, glucose 11; pH 7.4, 37° C.) according to the Langendorff method. A thread was attached to the apex of the heart, and then the end was connected to a tension transducer to determine contractile tension. The preparation was stabilized, and then myocardial ischemia was induced by reducing a perfusion pressure (for 45 minutes). After reperfusion for 30 min, the heart was liquefied in nitric acid, and ventricular total sodium content was determined by atomic absorption analysis. Contractile tension was measured during the experiment, and a recovery rate in the contractile tension at 30 min after the reperfusion, relative to the contractile tension at the beginning of the experiment, was used as an index of cardiac contractility.

(Results)

The results are shown in Table 3. In the table, ** represents p<0.01 vs. control by Dunnett's multiple comparison test, *** represents P<0.001 vs. control by Dunnett's multiple comparison test, ## represents P<0.01 vs. normal group by t-test, and ### represents P<0.001 vs. normal group by t-test.

In the ischemic and reperfused hearts, an increase in ventricular total sodium content (sodium overload) and a decrease in recovery in contractile tension were observed compared with those in the normal hearts. When MCC-135 was added in the reperfusion solution, the increase of ventricular sodium content, which was induced by ischemia and reperfusion, was suppressed, and the decrease in the recovery in contractile tension was improved. Amiloride (an inhibitor of sodium/proton exchanger, purchased from Sigma, St. Louis, Mo., USA) gave a decrease in recovery of contractile tension at a high dose, however, gave no effect on an increase of ventricular sodium content.

TABLE 3
Effects of MCC-135 on ventricular calcium content
and recovery in contractile tension

| | | | Recovery in |
|---|---|---|---|
| | | Sodium Content | Contractile Tension |
| Group | N | (μmol/g) | (%) |
| Normal | 9 | 59.4 ± 4.6 | 89.6 ± 2.0 |
| Control | 10 | 79.6 ± 5.1## | 25.0 ± 3.4### |
| MCC-135 10⁻⁹ M | 8 | 68.4 ± 6.2 | 40.0 ± 4.9 |
| MCC-135 10⁻⁸ M | 8 | 68.7 ± 3.7 | 49.1 ± 2.9 |
| MCC-135 10⁻⁷ M | 8 | 62.2 ± 0.6 | 68.4 ± 3.2** |
| MCC-135 10⁻⁶ M | 8 | 59.9 ± 2.3* | 79.4 ± 4.7*** |
| Amiloride 10⁻⁵ M | 8 | 77.9 ± 3.9 | 33.3 ± 7.2 |
| Amiloride 10⁻⁴ M | 8 | 71.8 ± 3.3 | 45.4 ± 3.8 |
| Amiloride 10⁻³ M | 8 | 64.8 ± 3.7 | 59.0 ± 4.7* |

## P < 0.01,
## P < 0.001 vs. normal
*P < 0.05,
**P < 0.001,
P < 0.001 vs. control

From the above results, the compound of the present invention was demonstrated to be effective in suppressing the increase in sodium ion content in cardiac myocytes induced by ischemia and reperfusion.

### INDUSTRIAL APPLICABILITY

According to the present invention, a medicament for suppressing intracellular excess accumulation of sodium ions is successfully provided. The medicament of the present invention is effective for therapeutic and or preventive treatment of disorders induced by clinically observed ischemia and reperfusion, or therapeutic and/or preventive treatment of cardiac disorders resulting from cardiosurgery operations. Further, because an intracellular excess accumulation of sodium ion will induce an intracellular excess accumulation of calcium ions, the medicament is effective for therapeutic and/or preventive treatment of cardiovascular diseases such as ischemic heart disease, heart failure, hypertension, arrhythmia and the like.

The whole content described in the specification of Japanese Patent Application No. 2002-255746, which is a basic Japanese patent application on which a priority is claimed for the present application, are incorporated by reference as a part of the disclosures in the present specification.

1. A medicament for suppressing intracellular excess accumulation of sodium ions, which comprises, as an active ingredient, an aminobenzenesulfonic acid derivative represented by the following general formula (I) or a salt thereof, or a hydrate thereof or a solvate thereof: 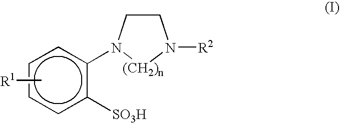
wherein R¹ represents hydrogen atom, a C₁-C₆ alkyl group, a C₃-C₇ cycloalkyl group, a halogenated C₁-C₄ alkyl group, a halogen atom, or a C₆-C₁₂ aryl group; R² represents hydrogen atom, a C₁-C₆ alkyl group, or a C₇-C₁₂ aralkyl group which may have one or more substituents selected from the group consisting of cyano group, nitro group, a C₁-C₆ alkoxyl group, a halogen atom, a C₁-C₆ alkyl group, and amino group; and n represents an integer of from 1 to 4.

2. The medicament for suppressing intracellular excess accumulation of sodium ions according to claim 1, which is used for therapeutic and/or preventive treatment of disorders resulting from ischemia and reperfusion.

3. The medicament for suppressing intracellular excess accumulation of sodium ions according to claim 1, which is characterized to suppress an increase of intracellular sodium content induced by ischemia and reperfusion.

4. The medicament for suppressing intracellular excess accumulation of sodium ions according to claim 1, wherein a substituting position R¹ is position-5.

5. The medicament for suppressing intracellular excess accumulation of sodium ions according to claim 1, wherein n is 2.

6. The medicament for suppressing intracellular excess accumulation of sodium ions according to claim 1, wherein R² is hydrogen atom, a C₁-C₃ alkyl group, or a C₇-C₁₂ aralkyl group which may have one or more substituents selected from the group consisting of a C₁-C₃ alkyl group, a C₁-C₃ alkoxyl group, and a halogen atom.

7. The medicament for suppressing intracellular excess accumulation of sodium ions according to claim 1, wherein R² is hydrogen atom or a C₇-C₁₂ aralkyl group which may have one or more C₁-C₃ alkoxyl groups.

8. The medicament for suppressing intracellular excess accumulation of sodium ions according to any claim 1, wherein R² is hydrogen atom.

9. The medicament for suppressing intracellular excess accumulation of sodium ions according to claim 1, wherein R¹ is hydrogen atom, a C₁-C₆ alkyl group, a C₅-C₆ cycloalkyl group, trifluoromethyl group, a halogen atom, or phenyl group.

10. The medicament for suppressing intracellular excess accumulation of sodium ions according to claim 1, wherein R¹ is a C₁-C₃ alkyl group, cyclohexyl group, trifluoromethyl group, chlorine atom, bromine atom, or phenyl group.

11. The medicament for suppressing intracellular excess accumulation of sodium ions according to claim 1, wherein R¹ is methyl group or propyl group.

12. The medicament for suppressing intracellular excess accumulation of sodium ions according to claim 1, wherein the active ingredient is selected from the following compounds:
5-methyl-2-(1-piperazinyl)benzenesulfonic acid;
5-trifluoromethyl-2-(1-piperazinyl)benzenesulfonic acid;
5-n-propyl-2-(1-piperazinyl)benzenesulfonic acid;
5-phenyl-2-(1-piperazinyl)benzenesulfonic acid;
5-chloro-2-(1-piperazinyl)benzenesulfonic acid;
5-bromo-2-(1-piperazinyl)benzenesulfonic acid;
5-isopropyl-2-(1-piperazinyl)benzenesulfonic acid;
5-cyclohexyl-2-(1-piperazinyl)benzenesulfonic acid;
5-n-propyl-2-(1-homopiperazinyl)benzenesulfonic acid;
5-n-propyl-2-[4-(2,3,4-trimethoxybenzyl)-1-piperazinyl]benzenesulfonic acid;
5-n-propyl-2-[4-(3,4-dimethoxybenzyl)-1-piperazinyl]benzenesulfonic acid or a salt thereof, or a hydrate thereof or a solvate thereof.

13. The medicament for suppressing intracellular excess accumulation of sodium ions according to claim 12, wherein the active ingredient is selected from the following compounds:
5-methyl-2-(1-piperazinyl)benzenesulfonic acid and 5-n-propyl-2-(1-piperazinyl)-benzenesulfonic acid
or a salt thereof, or a hydrate thereof or a solvate thereof.

14. The medicament for suppressing intracellular excess accumulation of sodium ions according to claim 1, wherein the active ingredient is 5-methyl-2-(1-piperazinyl)benzenesulfonic acid monohydrate.

15. A medicament for therapeutic and/or preventive treatment of diseases caused by intracellular excess accumulation of sodium ions (provided that an ischemic heart disease, heart failure, hypertension, and arrhythmia are excluded), which comprises, as an active ingredient, the medicament for suppressing intracellular excess accumulation of sodium ions according to claim 1.

16. A medicament for therapeutic and/or preventive treatment of cardiovascular diseases caused by intracellular excess accumulation of calcium ions that occurs successively after intracellular excess accumulation of sodium ions, which comprises, as an active ingredient, the medicament for suppressing intracellular excess accumulation of sodium ions according to claim 1.

17. The therapeutic and/or preventive medicament according to claim 16, wherein the cardiovascular diseases caused by intracellular excess accumulation of calcium ions that occurs successively after intracellular excess accumulation of sodium ions is an ischemic heart disease, heart failure, hypertension, or arrhythmia.

18. The therapeutic and/or preventive medicament according to claim 17, wherein the ischemic heart disease is myocardial infarct or angina pectoris.

19. A medicament for therapeutic and/or preventive treatment of cardiac disorders resulting from cardiosurgery operations, which comprises, as an active ingredient, an aminobenzenesulfonic acid derivative represented by the following general formula (I) or a salt thereof, or a hydrate thereof or a solvate thereof: 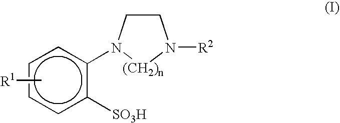
wherein R¹ represents hydrogen atom, a C₁-C₆ alkyl group, a C₃-C₇ cycloalkyl group, a halogenated C₁-C₄ alkyl group, a halogen atom, or a C₆-C₁₂ aryl group; R² represents hydrogen atom, a C₁-C₆ alkyl group, or a C₇-C₁₂ aralkyl group which may have one or more substituents selected from the group consisting of cyano group, nitro group, a C₁-C₆ alkoxyl group, a halogen atom, a C₁-C₆ alkyl group, and amino group; and n represents an integer of from 1 to 4.

20. The medicament for therapeutic and/or preventive treatment of cardiac disorders resulting from cardiosurgery operations according to claim 19, wherein a substituting position R¹ is position-5.

21. The medicament for therapeutic and/or preventive treatment of cardiac disorders resulting from cardiosurgery operations according to claim 19, wherein n is 2.

22. The medicament for therapeutic and/or preventive treatment of cardiac disorders resulting from cardiosurgery operations according to claim 19, wherein R² is hydrogen atom, a C₁-C₃ alkyl group, or a C₇-C₁₂ aralkyl group which may have one or more substituents selected from the group consisting of a C₁-C₃ alkyl group, a C₁-C₃ alkoxyl group, and a halogen atom.

23. The medicament for therapeutic and/or preventive treatment of cardiac disorders resulting from cardiosurgery operations according to claim 19, wherein R² is hydrogen atom or a C₇-C₁₂ aralkyl group which may have one or more C₁-C₃ alkoxyl groups.

24. The medicament for therapeutic and/or preventive treatment of cardiac disorders resulting from cardiosurgery operations according to claim 19, wherein R² is hydrogen atom.

25. The medicament for therapeutic and/or preventive treatment of cardiac disorders resulting from cardiosurgery operations according to claim 19, wherein R¹ is hydrogen atom, a C₁-C₆ alkyl group, a C₅-C₆ cycloalkyl group, trifluoromethyl group, a halogen atom, or phenyl group.

26. The medicament for therapeutic and/or preventive treatment of cardiac disorders resulting from cardiosurgery operations according to claim 19, wherein R¹ is a C₁-C₃ alkyl group, cyclohexyl group, trifluoromethyl group, chlorine atom, bromine atom, or phenyl group.

27. The medicament for therapeutic and/or preventive treatment of cardiac disorders resulting from cardiosurgery operations according to claim 19, wherein R¹ is methyl group or propyl group.

28. The medicament for therapeutic and/or preventive treatment of cardiac disorders resulting from cardiosurgery operations according to claim 19, wherein the active ingredient is selected from the following compounds:
5-methyl-2-(1-piperazinyl)benzenesulfonic acid;
5-trifluoromethyl-2-(1-piperazinyl)benzenesulfonic acid;
5-n-propyl-2-(1-piperazinyl)benzenesulfonic acid;
5-phenyl-2-(1-piperazinyl)benzenesulfonic acid;
5-chloro-2-(1-piperazinyl)benzenesulfonic acid;
5-bromo-2-(1-piperazinyl)benzenesulfonic acid;
5-isopropyl-2-(1-piperazinyl)benzenesulfonic acid;
5-cyclohexyl-2-(1-piperazinyl)benzenesulfonic acid;
5-n-propyl-2-(1-homopiperazinyl)benzenesulfonic acid;
5-n-propyl-2-[4-(2,3,4-trimethoxybenzyl)-1-piperazinyl]benzenesulfonic acid;
5-n-propyl-2-[4-(3,4-dimethoxybenzyl)-1-piperazinyl]benzenesulfonic acid or a salt thereof, or a hydrate thereof or a solvate thereof.

29. The medicament for therapeutic and/or preventive treatment of cardiac disorders resulting from cardiosurgery operations according to claim 28, wherein the active ingredient is selected from the following compounds:
5-methyl-2-(1-piperazinyl)benzenesulfonic acid and 5-n-propyl-2-(1-piperazinyl)-benzenesulfonic acid
or a salt thereof, or a hydrate thereof or a solvate thereof.

30. The medicament for therapeutic and/or preventive treatment of cardiac disorders resulting from cardiosurgery operations according to claim 19, wherein the active ingredient is
5-methyl-2-(1-piperazinyl)benzenesulfonic acid monohydrate.
